(19) United States
(12) Patent Application Publication
(10) Pub. No.: US 2014/0004153 A1
(43) Pub. Date: Jan. 2, 2014

(54) USE OF SOMATIC MUTATIONS IN THE EXTRACELLULAR DOMAIN OF TRANSMEMBRANE PROTEINS IN A PATIENT'S TUMOR AS IMMUNOGENS FOR ACTIVE HUMORAL IMMUNOTHERAPY OF CANCER

(71) Applicant: LANCELL, L.L.C., SAN DIEGO, CA (US)

(72) Inventors: CAROL O COWING, DEL MAR, CA (US); CHRISTOPHER COWING-ZITRON, DEL MAR, CA (US)

(21) Appl. No.: 13/932,649

(22) Filed: Jul. 1, 2013

Related U.S. Application Data
(60) Provisional application No. 61/667,359, filed on Jul. 2, 2012.

(51) Int. Cl. G01N 33/68

(52) U.S. Cl. CPC G01N 33/6893
USPC 424/277.1; 435/6.11

(57) ABSTRACT

Methods of identifying tumor-specific mutations that can serve as B cell epitopes for the induction of autologous tumor-specific antibodies are disclosed. In some embodiments, the methods include: identifying a tumor-specific mutation in an expressed gene of a subject having cancer; determining that the mutation is in a region of an open reading frame predicted to encode an extracellular domain of a membrane protein; and identifying a corresponding mutant polypeptide encoded by the expressed gene comprising the tumor-specific mutation. Some embodiments relate to a method of inducing a tumor specific immune response in a subject including administering one or more polypeptides identified according to the methods disclosed herein to a subject. Other embodiments relate to pharmaceutical compositions including mutant polypeptide(s).

### RELATED APPLICATIONS

This application claims benefit of U.S. Provisional Application No. 61/667,359 filed Jul. 2, 2012, which is hereby incorporated by reference in its entirety.

### FIELD OF THE INVENTION

The invention relates to the identification of tumor antigens and use of the tumor antigens to produce an immune response in an animal.

### BACKGROUND OF THE INVENTION

In 1909, Paul Ehrlich proposed that tumor cells are a common occurrence and are usually eliminated by the immune system (Kim, R. et al. 2007 Immunology 121(1):1-14). While prescient, his hypothesis was untestable; how does one measure the absence of something that was too small to detect when it was there? Now, more than a century later, most investigators are convinced that the immune system does eliminate tumors because congenital, acquired and induced loss of immune function is accompanied by an increased incidence of tumors in humans and laboratory animals (Kim, R. et al. 2007 Immunology 121(1):1-14; Yuhas, J. M. et al. 1974 Cancer research 34(4):722-8; Dunn, G. P. et al. 2002 Nat Immunol 3(11): 991-888; Cramer, D. W. and Finn O. J. 2011 Current Opinion in Immunology 23(2): 265-271; Fridman, W. H. et al. 2011 Current Opinion in Immunology 23(2): 272-278; Shapiro, R. S. 2011 American Journal of Hematology 86(1):48-55; and Vesely, M. D. et al. 2011 Annu Rev Immunol 29: 235-271). Moreover when immune surveillance fails, there is now genomic evidence indicating that immune responses have selected or “edited” surviving tumor cells (Matsushita, H. et al. 2012 Nature 482(7385):400-404).

### SUMMARY OF THE INVENTION

Disclosed herein are methods to identify tumor-specific mutations that can serve as B cell epitopes for the induction of autologous tumor-specific antibodies.

In some embodiments, the method of identifying a tumor-specific antigen comprises: identifying a tumor-specific mutation in an expressed gene of a subject having cancer; determining that said mutation is in a region of an open reading frame predicted to encode an extracellular domain of a membrane protein; and identifying a corresponding mutant polypeptide encoded by the expressed gene comprising the tumor-specific mutation.

In some embodiments, the mutation is a single nucleotide polymorphism (SNP).

In some embodiments, the mutant polypeptide is about 6-10 amino acids in length.

In some embodiments, the mutant polypeptide is greater than 10 amino acids in length.

In some embodiments, the mutant polypeptide is greater than 15 amino acids in length.

In some embodiments, the mutant polypeptide is greater than 20 amino acids in length.

In some embodiments, the mutant polypeptide is greater than 30 amino acids in length.

In some embodiments, the method further comprising selecting a polypeptide identified in step (c) that activates an antibody response.

Some embodiments relate to a method of inducing a tumor specific immune response in a subject comprising administering one or more polypeptides identified according to the methods disclosed herein to said subject.

Some embodiments relate to a pharmaceutical composition comprising a mutant polypeptide identified according to the methods disclosed herein and a pharmaceutically acceptable carrier.

### DETAILED DESCRIPTION OF THE PREFERRED EMBODIMENT

Numerous attempts have been made to coax the immune system to destroy tumors that had escaped immune surveillance. Virtually all have failed. There are presently 75 licensed vaccines for infectious diseases (FDA, 2012 FDA approved vaccines 2012. Available on the World-Wide-Web at: fda.gov/BiologicsBloodVaccines/Vaccines/ApprovedProducts/ucm093833.htm), but only two therapeutic cancer vaccines: 1) Sipuleucel-T (PROVENGE®) for prostate cancer, which prolonged life by less than 4 months in Phase 3 clinical trials (Kantoff, P. W. et al. 2010 New England Journal of Medicine 363(5):411-422) and costs $93,000 per patient; and 2) Ipilimumab (YERVOY®) for melanoma, with similar efficacy in Phase 3 trials (YERVOY® (Ipilimumab) package insert. Princeton, N.J.: Bristol-Myers Squibb; March 2011) and a reputed cost of $120,000. If the immune system destroys some emerging tumors, why can't it be induced to destroy all tumors? The failure cannot be ascribed to a lack of effort, lack of funding, or absence of the ability to design effective vaccines. Clearly, there are fundamental barriers to active tumor immunotherapy. Historically, the success rate for development of tumor vaccines is far lower than the success rate for development of infectious disease vaccines. This is evident from a search of the National Center for Biotechnology Information (NCBI) PubMed database, summarized in the following table:

| | | | | Success Rate |
|---|---|---|---|---|
| Pub Med | | | Vaccines | (Vaccine/ |
| Search Term | Publications | Years | Developed | Publication) |
| Infectious | 13,483 | 1900-2012 | 75 | 1 per 180 |
| disease | | | | |
| vaccines | | | | |
| Tumor | 54,609 | 1951-2012 | 2 | 1 per 27,305 |
| immunotherapy | | | | |

### Barriers to Active Tumor Immunotherapy

Immunity is analogous to a tightrope. On one side, failure to recognize an infecting pathogen can result in being overtaken by microorganisms that multiply faster than our cells. If HIV had acquired aerosol transmissibility and anti-retroviral drugs had not been developed, the loss of human life could have been catastrophic because infection does not induce sterilizing immunity, and vaccines have eluded all efforts to date. On the opposite side of the paradigm, mistaking oneself for a pathogen can cause chronic autoimmune disease. Even more dangerous is over-reacting to a real pathogen, which can be rapidly fatal. We have only recently realized the extent of the natural mechanisms that prevent excessive immune responses. Tumors have exploited this side of the paradigm to evade persistent efforts to develop vaccines.

Tumor antigen-specific T lymphocytes induced by numerous methods can destroy tumor cells ex vivo, but fail to alter the clinical course of disease. Tumor cells can escape immune destruction by antigen loss (Seliger, B. et al. 2001 Cancer Research 61(24): 8647-8650; Seliger, B. et al. Cancer Research 61(3):1095-9; and Montagut, C et al. 2012 Nature Medicine 18(2):221-3). Those tumor cells that survive immune surveillance have also acquired an astonishing number of immunosuppressive mechanisms (Biragyn, A. and Longo, D. L. 2012 Semin Cancer Biol 22(1):50-9) that enable the tumor to prevent natural induction of anti-tumor immunity (Scarlett, U.K. et al. 2012 J Exp Med 209(3): 495-506) and block attack by artificially induced or expanded tumor-specific T cells (Tan, W. et al. 2011 Nature 470(7335): 548-53; Romano, E. et al. 2011 Clinical Cancer Research 17(7): 1984-1997; and Papatriantafyllou, M. 2011 Nature Reviews Immunology 11(4):236-7). The strong resistance of tumor cells to T cell-mediated attack is apparent from initial failures to genetically engineer T cells that could overcome this barrier (Urba, W. J. and Longo, D. L. 2011 The New England Journal of Medicine 365(8):754-7). Then, when second and third generation constructs succeeded in producing a T cell that could withstand the tumor microenvironment, it became clear that an inducible suicide gene would have to be added to eliminate those T cells after they had killed the tumor but continued to attack the host (Urba, W. J. and Longo, D. L. 2011 The New England Journal of Medicine 365(8):754-7; Kalos, M. et al. 2011 Science Translational Medicine 3(95):95ra73; Porter, D. L. et al. 2011 The New England Journal of Medicine 365(8):725-33; and Hoyos, V. et al. 2010 Leukemia 24(6):1160-70).

An alternate approach, blocking CTLA-4 expression on regulatory T cells induced by tumors, was seen as a breakthrough that would revitalize active tumor immunotherapy (Pardoll, D. and Drake, C. 2012 J Exp Med 209(2):201-9; and Mellman, I. et al. 2011 Nature 480(7378):480-9). CTLA-4 is a competitive inhibitor of co-stimulation of T cells through CD28. To circumvent exploitation of this mechanism by tumors, an anti-CTLA-4 mAb was combined with a gp100 peptide vaccine in melanoma patients. Only 20% of patients treated with the vaccine plus anti-CTLA-4 had a greater response than those treated with the vaccine alone, and this led to an average of 4 months increased survival. At the same dose of anti-CTLA-4, 15% of patients experienced “severe to fatal immune-mediated adverse reactions” (Yervoy (ipilimumab) package insert. Princeton, N.J.: Bristol-Myers Squibb; March 2011). This toxicity is not surprising considering the phenotype of CTLA-4 knock-out mice: “death in 3 wk from destructive lymphoid infiltration into multiple organs (Tivol, E. A. et al. 1995 Immunity 3(5):541-7; and Waterhouse, P. et al. 1995 Science 270(5238):985-8), attesting to its critical role as a regulator of T cell-dependent immune responses” (Pardoll, D. and Drake, C. 2012 J Exp Med 209(2):201-9). CTLA-4 is not unique. Tumors did not invent these immunosuppressive mechanisms but co-opted them from normal controls that prevent excessive immune responses leading to systemic shock and multi-organ failure. The danger inherent in counteracting these mechanisms is evident from the numerous life-threatening adverse events that have occurred subsequent to clinical use of treatments that override immune controls (Gaston, R. S. et al. 1991 Kidney International 39(1):141-8; Wing, M. G. et al. 1996 The Journal of Clinical Investigation 98(12):2819-26; Winkler, U. et al. 1999 Blood 94(7):2217-24; Suntharalingam, G. et al. 2006 The New England Journal of Medicine 355(10):1018-28; Ascierto, P. A. et al. 2011 Journal of Translational Medicine 9:196; Brentjens, R. et al. 2010 Molecular Therapy 18(4):666-8; and Morgan, R.A. et al. 2010 Molecular Therapy 18(4):843-51). The next intervention, i.e., anti-PD-1, may have a better therapeutic index than anti-CTLA-4, and inhibitors of multiple tumor immunosuppressive mechanisms may be more effective than one. Unfortunately, the extent of tumor subversion of normal immune control mechanisms (Biragyn, A. and Longo, D. L. 2012 Semin Cancer Biol 22(1):50-9) suggests that inhibiting enough of these to produce broadly effective immunotherapy is likely to cause levels of toxicity that will prevent widespread use.

### Efforts to Overcome Barriers to Active Tumor Immunotherapy

The immunosuppressive tumor microenvironment blocks cell-mediated immunity but does not block the efficacy of anti-tumor antibodies (Ab). Dozens of mAbs currently in clinical use confirm this (Immunogenetics. Monoclonal antibodies approved and in use 2012 2012. Available on the World-Wide-Web at imgt.org/mAb-DB/index#Approval_antibodies). Moreover, it has been known for more than three decades that a subset of cancer patients develops anti-tumor Abs (Shiku, H. et al. 1976 J Exp Med 144(4):873-81). At the time, it was difficult to determine if those Abs were specific for the patient's tumor and whether they had any clinical significance. Primary human tumors did not readily adapt to tissue culture (Watanabe, T. et al. 1982 J Exp Med 156(6):1884-9), and the severely immunocompromised mice required to support the growth of primary xenogeneic tumor cells did not yet exist (Jax). Of necessity, human anti-tumor Abs were studied on allogeneic tumor cell lines, but this was not considered a problem at the time because investigators were seeking common tumor antigens. Today, these naturally occurring “autoantibodies” are being tested as a potential early diagnostic of cancer (Desmetz, C. et al. 2011 Journal of Cellular and Molecular Medicine 15(10):2013-24; and Boyle, P. et al. 2010 Annals of Oncology 22(2):383-9) and used as a source of tumor antigens for the induction of T cell mediated immunity (Zhou, S. et al. 2012 Combinatorial Chemistry & High Throughput Screening 15(3):202-15).

In the last few years, next generation sequencing (NGS) of tumor cell : normal cell pairs has revealed the molecular characteristics of tumors that confer growth advantage and those that enable tumors to resist immunity induced against common tumor antigens. NGS has identified numerous single nucleotide polymorphisms (SNPs) in the coding regions (exomes) of tumor cells. While several tumor types have a non-random distribution of mutations in particular oncogenes, the vast majority of tumor-specific somatic point mutations, ˜98%, are unique to each tumor (Matsushita, H. et al. 2012 Nature 482(7385):400-404).

While this genomic information may explain the clinical failure of immunotherapy based on common tumor antigens, what about whole autologous tumor cells, tumor lysates or total RNA? These immunogens express all of the products of somatic mutations in an individual's tumor; and yet, several immunotherapies based on this approach have not succeeded in Phase 3 clinical trials. One explanation fits the data. Activation of a naïve T cell is dependent on the frequency of cognate peptide : MHC complexes displayed on a dendritic cell (DC) plasma membrane and the expression of the requisite costimulatory molecules. If the frequency of a peptide : MHC complex is below the threshold level required for sustained signaling of a peptide-specific T cell, no activation will occur (Henrickson, S. E. et al. 2007 Current Opinion in Immunology 19(3):249-58; and Kuhns, M. S. and Davis, M. M. 2012 Frontiers in Immunology 3: 159). The problem with whole tumor cells, lysates or total RNA is that any one mutated peptide will be a needle in a haystack. DC' s have no mechanism for distinguishing a mutated from a wild-type peptide. All proteins will be processed for potential expression in an MHC groove, leading to such a low frequency of any one peptide : MHC complex that the chances for activating corresponding peptide-specific naive T cells may be vanishingly low.

On the other hand, the ability to identify expressed missense msSNPs in a patient's tumor offers the possibility to construct patient-specific cancer vaccines that target only the tumor and are immunogenic because the antigens are not expressed in normal tissue. As proof of concept, tumor cell exome msSNPs were recently used as a source of MHC class I restricted T cell epitopes for successful immunotherapy of the murine B16 melanoma (Castle, J. C. et al. 2012 Cancer Research 72(5):1081-91). This approach is also the subject of a recent patent application (WO 2011/143656A2). But, whole exome NGS technology may also offer a completely new approach to active tumor immunotherapy that will be unaffected by the immunosuppressive tumor microenvironment: induction of autologous tumor-specific Abs.

While it is incontrovertible that T cells and NK cells can kill tumor cells, it is equally well-established that anti-tumor Abs can destroy or retard the growth of tumor cells (Immunogenetics. Monoclonal antibodies approved and in use 2012 2012. Available on the World-Wide-Web at imgtorg/mAb-DB/index#Approval_antibodies). These mAbs are directed against over-expressed normal molecules rather than tumor-specific antigens, so the antigens will not induce active immunotherapy. But, the identification of expressed somatic mutations in tumors enables the identification of potential tumor-specific Ab targets. If these tumor-specific antigens exist, why don't tumors induce the relevant Abs? There is currently no direct evidence for or against this possibility. However, logic would suggest that any tumor cell that possesses the attributes of a DC, enabling it to activate naïve Th cells and induce tumor-specific Abs, is unlikely to survive long enough to cause disease and would constitute “successful tumor immuno surveillance.”

To our knowledge, the feasibility of using somatic mutations in the extracellular domain (ECD) of plasma membrane proteins on tumor cells as the source of immunogens for active induction of tumor-specific Ab-mediated immunotherapy has not been tested or even suggested. Extensive clinical experience has demonstrated the ability of anti-tumor mAbs to penetrate the immunosuppressive microenvironment of tumors and elicit a therapeutic response. We disclose herein methods to identify expressed tumor-specific somatic mutations. These mutated sequences provide a basis for constructing Ab-based therapeutic cancer vaccines that are non-toxic and cost-effective.

In some embodiments, a mutant peptide identified by the methods disclosed herein is about 6-10 amino acids in length. In another aspect, the mutant peptide is about 6-50 amino acids in length. For example, a mutant peptide may be greater than 10 amino acids in length, greater than 15 amino acids in length, greater than 20 amino acids in length or greater than 30 amino acids in length. In some embodiments, the mutant peptides is about 24-40 amino acids in length.

A subject for which a tumor specific immune response is generated may be any mammal, e.g., a human, dog, cat, or horse. The cancer may be selected from the non-limiting group consisting of breast cancer, ovarian cancer, prostate cancer, lung cancer, kidney cancer, gastric cancer, colon cancer, testicular cancer, head and neck cancer, pancreatic cancer, brain cancer, melanoma lymphoma, such as B-cell lymphoma or leukemia, such as acute myelogenous leukemia, chronic myelogenous leukemia, chronic lymphocytic leukemia, or T cell lymphocytic leukemia.

Also included in the invention are pharmaceutical compositions containing a peptide or polypeptide identified according the methods of the invention and a pharmaceutically acceptable carrier.

One of the critical barriers to developing curative and tumor-specific immunotherapy is the identification and selection of highly restricted tumor antigens to avoid autoimmunity. Tumor antigens, which arise as a result of genetic change within malignant cells, represent the most tumor-specific class of antigens. Tumor antigens have rarely been used in vaccines due to technical difficulties in identifying them. Disclosed herein are methods to identify tumor antigens based on a mutation in an extracellular domain of a membrane protein.

The present invention is based, on the identification of certain mutations (e.g., the variants or alleles that are present in cancer cells). In particular, these mutations are present in the genome of cancer cells of a subject having cancer but not in normal tissue from the subject.

Genetic mutations in tumors would be considered useful for the immunological targeting of tumors if they lead to changes in the amino acid sequence of an extracellular domain of a membrane protein exclusively in the tumor. Useful mutations include: (1) non-synonymous mutations leading to different amino acids in the protein; (2) read-through mutations in which a stop codon is modified or deleted, leading to translation of a longer protein with a novel tumor-specific sequence at the C-terminus; (3) splice site mutations that lead to the inclusion of an intron in the mature mRNA and thus a unique tumor-specific protein sequence; (4) chromosomal rearrangements that give rise to a chimeric protein with tumor-specific sequences at the junction of 2 proteins (i.e., gene fusion); and (5) frameshift mutations or deletions that lead to a new open reading frame with a novel tumor-specific protein sequence.

Peptides with mutations or mutated polypeptides arising from for example, splice-site, frameshift, read-through, or gene fusion mutations in tumor cells may be identified by sequencing DNA, RNA or protein in tumor versus normal cells.

Also within the scope of the inventions are peptides including previous identified tumor specific mutations.

### Tumor Peptides

The invention further includes isolated peptides that comprise the tumor specific mutations identified by the methods of the invention, peptides that comprise know tumor specific mutations, and mutant polypeptides or fragments thereof identified by the method of the invention. The term “peptide” is used interchangeably with “mutant peptide” in the present specification to designate a series of residues, typically L-amino acids, connected one to the other, typically by peptide bonds between the α-amino and carboxyl groups of adjacent amino acids. Similarly, the term “polypeptide” is used interchangeably with “mutant polypeptide” in the present specification to designate a series of residues, typically L-amino acids, connected one to the other, typically by peptide bonds between the α-amino and carboxyl groups of adjacent amino acids. The polypeptides or peptides can be a variety of lengths, either in their neutral (uncharged) forms or in forms which are salts, and either free of modifications such as glycosylation, side chain oxidation, or phosphorylation or containing these modifications, subject to the condition that the modification not destroy the biological activity of the polypeptides as herein described.

In certain embodiments, the size of the at least one antigenic tumor peptide molecule may comprise, but is not limited to, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, about 70, about 80, about 90, about 100, about 110, about 120 or greater amino acid residues, and any range derivable therein. In specific embodiments the antigenic peptide molecules are equal to or less than 50 amino acids.

The antigenic peptides and polypeptides preferably do not induce an autoimmune response and/or invoke immunological tolerance when administered to a subject.

The invention also provides compositions comprising at least one or more antigenic peptides. In some embodiments the composition contains at least two distinct peptides. Preferably, the at least two distint peptides are derived from the same polypeptide. By distinct polypeptides is meant that the peptide vary by length, amino acid sequence or both. The peptides are derived from any polypeptide know to or have been found to by the methods of the invention to contain a tumor specific mutation.

The antigenic peptides and polypeptides can also be modified by extending or decreasing the compound's amino acid sequence, e.g., by the addition or deletion of amino acids. The peptides, polypeptides or analogs can also be modified by altering the order or composition of certain residues, it being readily appreciated that certain amino acid residues essential for biological activity, e.g., those at critical contact sites or conserved residues, may generally not be altered without an adverse effect on biological activity. The non-critical amino acids need not be limited to those naturally occurring in proteins, such as L-α-amino acids, or their D-isomers, but may include non-natural amino acids as well, such as β-γ-δ-amino acids, as well as many derivatives of L-α-amino acids.

Amino acid substitutions are typically of single residues. Substitutions, deletions, insertions or any combination thereof may be combined to arrive at a final peptide. Substitutional variants are those in which at least one residue of a peptide has been removed and a different residue inserted in its place.

Proteins or peptides may be made by any technique known to those of skill in the art, including the expression of proteins, polypeptides or peptides through standard molecular biological techniques, the isolation of proteins or peptides from natural sources, or the chemical synthesis of proteins or peptides. The nucleotide and protein, polypeptide and peptide sequences corresponding to various genes have been previously disclosed, and may be found at computerized databases known to those of ordinary skill in the art. One such database is the National Center for Biotechnology Information's Genbank and GenPept databases located at the National Institutes of Health website. The coding regions for known genes may be amplified and/or expressed using the techniques disclosed herein or as would be known to those of ordinary skill in the art. Alternatively, various commercial preparations of proteins, polypeptides and peptides are known to those of skill in the art.

In a further aspect of the invention provides a nucleic acid (e.g., polynucleotide) encoding a antigenic peptide of the invention. The polynucleotide may be e.g., DNA, cDNA, PNA, CNA, RNA, either single- and/or double-stranded, or native or stabilized forms of polynucleotides, such as e.g., polynucleotides with a phosphorothiate backbone, or combinations thereof and it may or may not contain introns so long as it codes for the peptide. Of course, only peptides that contain naturally occurring amino acid residues joined by naturally occurring peptide bonds are encodable by a polynucleotide. A still further aspect of the invention provides an expression vector capable of expressing a polypeptide according to the invention. Expression vectors for different cell types are well known in the art and can be selected without undue experimentation. Generally, the DNA is inserted into an expression vector, such as a plasmid, in proper orientation and correct reading frame for expression. If necessary, the DNA may be linked to the appropriate transcriptional and translational regulatory control nucleotide sequences recognized by the desired host, although such controls are generally available in the expression vector. The vector is then introduced into the host through standard techniques.

### Vaccine Compositions

The present invention is directed to an immunogenic composition, e.g., a vaccine composition capable of raising a specific antibody response. The vaccine composition comprises mutant peptides and mutant polypeptides corresponding to tumor specific antigens identified by the methods described herein.

A suitable vaccine will preferably contain between 1 and 20 peptides, more preferably 2, 3, 4, 5, 6, 7, 8, 9, 10, 11, 12, 13, 14, 15, 16, 17, 18, 19, or 20 different peptides, further preferred 6, 7, 8, 9, 10 11, 12, 13, or 14 different peptides, and most preferably 12, 13 or 14 different peptides.

The vaccine composition can further comprise an adjuvant. Adjuvants are any substance whose admixture into the vaccine composition increases or otherwise modifies the immune response to the mutant peptide. The ability of an adjuvant to increase the immune response to an antigen is typically manifested by a significant increase in immune-mediated reaction, or reduction in disease symptoms. For example, an increase in humoral immunity is typically manifested by a significant increase in the titer of antibodies raised to the antigen.

. Particular adjuvant compositions and methods of use are well known and not particularly limiting. Typical adjuvants and strategies are described for example in U.S. Pat. No. 7,229,621 and in U.S. Application Publication No. 2011/0293637, the entire disclosures of which are incorporated herein in their entireties.

A vaccine composition according to the present invention may comprise more than one different adjuvants. It is also contemplated that the peptide or polypeptide, and the adjuvant can be administered separately in any appropriate sequence.

### Therapeutic Methods

The invention further provides a method of inducing a tumor specific immune response in a subject, vaccinating against a tumor, treating and or alleviating a symptom of cancer in a subject by administering the subject an antigenic peptide or vaccine composition of the invention.

The subject has been diagnosed with cancer or is at risk of developing cancer. In some embodiments, the subject is a human, dog, cat, horse or any animal in which a tumor specific immune response is desired. The tumor is any solid tumor, such as breast, ovarian, prostate, lung, kidney, gastric, colon, testicular, head and neck, pancreas, brain, melanoma, and other tumors of tissue organs and hematological tumors, such as lymphomas and leukemias, including acute myelogenous leukemia, chronic myelogenous leukemia, chronic lymphocytic leukemia, T cell lymphocytic leukemia, and B cell lymphomas.

The antigenic peptide, polypeptide or vaccine composition of the invention can be administered alone or in combination with other therapeutic agents. A therapeutic agent is for example, a chemotherapeutic agent, radiation, or immunotherapy. Any suitable therapeutic treatment for a particular cancer may be administered.

The optimum amount of each peptide to be included in the vaccine composition and the optimum dosing regimen can be determined by one skilled in the art without undue experimentation. For example, the peptide or its variant may be prepared for intravenous (i.v.) injection, sub-cutaneous (s.c.) injection, intradermal (i.d.) injection, intraperitoneal (i.p.) injection, intramuscular (i.m.) injection. Preferred methods of peptide injection include s.c., i.d., i.p., i.m., and i.v. Preferred methods of DNA injection include i.d., i.m., s.c., i.p. and i.v. For example, doses of between 1 and 500 mg 50 μg and 1.5 mg, preferably 125 μg to 500 μg, of peptide or DNA may be given and will depend from the respective peptide or DNA. Other methods of administration of the vaccine composition are known to those skilled in the art.

For a composition to be used as a vaccine for cancer, peptides whose endogenous parent proteins are expressed in high amounts in normal tissues will be avoided or be present in low amounts in the composition of the invention. On the other hand, if it is known that the tumor of a patient expresses high amounts of a certain protein, the respective pharmaceutical composition for treatment of this cancer may be present in high amounts and/or more than one peptide specific for this particularly protein or pathway of this protein may be included.

Pharmaceutical compositions comprising the peptide of the invention may be administered to an individual already suffering from cancer. In therapeutic applications, compositions are administered to a patient in an amount sufficient to elicit an effective antibody response to the tumor antigen and to cure or at least partially arrest symptoms and/or complications. An amount adequate to accomplish this is defined as “therapeutically effective dose.” Amounts effective for this use will depend on, e.g., the peptide composition, the manner of administration, the stage and severity of the disease being treated, the weight and general state of health of the patient, and the judgment of the prescribing physician, but generally range for the initial immunization (that is for therapeutic or prophylactic administration) from about 1.0 μg to about 50,000 μg of peptide for a 70 kg patient, followed by boosting dosages or from about 1.0 μg to about 10,000 μg of peptide pursuant to a boosting regimen over weeks to months depending upon the patient's response and condition by measuring specific antibody activity in the patient's blood.

For therapeutic use, administration may begin at the detection or surgical removal of tumors. This may be followed by boosting doses until at least symptoms are substantially abated and for a period thereafter.

The pharmaceutical compositions (e.g., vaccine compositions) for therapeutic treatment are intended for parenteral, topical, nasal, oral or local administration. Preferably, the pharmaceutical compositions are administered parenterally, e.g., intravenously, subcutaneously, intradermally, or intramuscularly. The compositions may be administered at the site of surgical exiscion to induce a local immune response to the tumor. The invention provides compositions for parenteral administration which comprise a solution of the peptides and vaccine compositions are dissolved or suspended in an acceptable carrier, preferably an aqueous carrier. A variety of aqueous carriers may be used, e.g., water, buffered water, 0.9% saline, 0.3% glycine, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

The concentration of peptides of the invention in the pharmaceutical formulations can vary widely, i.e., from less than about 0.1%, usually at or at least about 2% to as much as 20% to 50% or more by weight, and will be selected primarily by fluid volumes, viscosities, etc., in accordance with the particular mode of administration selected.

The peptide of the invention may also be administered via liposomes, which target the peptides to a particular cells tissue, such as lymphoid tissue. Liposomes are also useful in increasing the half-life of the peptides. Liposomes include emulsions, foams, micelles, insoluble monolayers, liquid crystals, phospholipid dispersions, lamellar layers and the like.

For targeting to immune cells, a ligand to be incorporated into the liposome can include, e.g., antibodies or fragments thereof specific for cell surface determinants of the desired immune system cells. A liposome suspension containing a peptide may be administered intravenously, locally, topically, etc. in a dose which varies according to, inter alia, the manner of administration, the peptide being delivered, and the stage of the disease being treated.

For solid compositions, conventional or nanoparticle nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more peptides of the invention, and more preferably at a concentration of 25%-75%.

For aerosol administration, the immunogenic peptides are preferably supplied in finely divided form along with a surfactant and propellant. Typical percentages of peptides are 0.01%-20% by weight, preferably 1%-10%. The surfactant must, of course, be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, preferably 0.25-5%. The balance of the composition is ordinarily propellant. A carrier can also be included as desired, as with, e.g., lecithin for intranasal delivery.

For therapeutic or immunization purposes, nucleic acids encoding the peptide of the invention and optionally one or more of the peptides described herein can also be administered to the patient. A number of methods are conveniently used to deliver the nucleic acids to the patient. For instance, the nucleic acid can be delivered directly, as “naked DNA.” The nucleic acids can also be administered using ballistic delivery. Particles comprised solely of DNA can be administered. Alternatively, DNA can be adhered to particles, such as gold particles.

The nucleic acids can also be delivered complexed to cationic compounds, such as cationic lipids.

The peptides and polypeptides of the invention can also be expressed by attenuated viral hosts, such as vaccinia or fowlpox. This approach involves the use of vaccinia virus as a vector to express nucleotide sequences that encode the peptide of the invention. Upon introduction into an acutely or chronically infected host or into a noninfected host, the recombinant vaccinia virus expresses the immunogenic peptide, and thereby elicits a host antibody response. A wide variety of other vectors useful for therapeutic administration or immunization of the peptides of the invention, e.g., Salmonella typhi vectors and the like, will be apparent to those skilled in the art from the description herein.

Standard regulatory sequences well known to those of skill in the art are included in the vector to ensure expression in the target cells. Several vector elements are required: a promoter with a down-stream cloning site for minigene insertion; a polyadenylation signal for efficient transcription termination; an E. coli origin of replication; and an E. coli selectable marker (e.g., ampicillin or kanamycin resistance). Numerous promoters can be used for this purpose, e.g., the human cytomegalovirus (hCMV) promoter.

Once an expression vector is selected, the minigene is cloned into the polylinker region downstream of the promoter. This plasmid is transformed into an appropriate E. coli strain, and DNA is prepared using standard techniques. The orientation and DNA sequence of the minigene, as well as all other elements included in the vector, are confirmed using restriction mapping and DNA sequence analysis. Bacterial cells harboring the correct plasmid can be stored as a master cell bank and a working cell bank.

Purified plasmid DNA can be prepared for injection using a variety of formulations. The simplest of these is reconstitution of lyophilized DNA in sterile phosphate-buffer saline (PBS). A variety of methods have been described, and new techniques may become available. As noted above, nucleic acids are conveniently formulated with cationic lipids. In addition, glycolipids, fusogenic liposomes, peptides and compounds referred to collectively as protective, interactive, non-condensing (PINC) could also be complexed to purified plasmid DNA to influence variables such as stability, intramuscular dispersion, or trafficking to specific organs or cell types.

### Identification of Targets

Our initial goal was to determine whether carcinogen-induced human tumors are likely to yield at least 3 to 5 effective targets for induction of autologous Ab-mediated immunotherapy. Several criteria must be met for tumor-specific mutations to qualify as immunogens for Ab-mediated immunotherapy.

To be accessible to Ab, tumor mutations must be in the ECD of transmembrane proteins. Despite the large number of human tumors that are estimated to have undergone NGS sequencing (˜2000), the frequency of somatic mutations occurring in tumor ECDs is unknown. The impetus for sequencing tumor cell:normal cell pairs was to find drugable, oncogenic or driver mutations. These were anticipated to occur primarily in catalytic domains of protein kinases, in DNA repair genes, tumor suppressor genes and other known oncogenes. Consequently, ECD sequences have often been ignored and rarely published. Fortunately, enough ECD mutations have been reported (Montagut, C et al. 2012 Nature Medicine 18(2):221-3, Letard, S. et al. 2008 Molecular Cancer Research 6(7):1137-45; Idbaih, A. et al. 2009 Neuropathology and Applied Neurobiology 35(2):208-13; Yang, Y. et al. 2010 Blood 116(7):1114-23; Lux, M. L. et al. 2000 The American Journal of Pathology 156(3):791-5; Zang, Z. J. et al. 2012 Nature Genetics 44(5):570-4; Berx, G. et al. 1996 Oncogene 13(9):1919-25; and Prickett, T. D. et al. 2009 Nature Genetics 41(10):1127-32) to permit the conclusion that human tumor mutations occur apparently randomly among protein domains (Berx, G. et al. 1996 Oncogene 13(9):1919-25), and when a transmembrane protein has a relatively large ECD, the frequency of mutations in the ECD is correspondingly greater than that in smaller domains, e.g., ERBB4 in melanoma (Prickett, T. D. et al. 2009 Nature Genetics 41(10):1127-32). Thus, while an accurate frequency of ECD somatic mutations in a particular tumor cell type will require mining Whole Exome Sequencing (WES) databases, ECD mutations seem to occur with approximately random frequency. This conclusion is supported by the frequency of somatic mutations found in plasma membrane proteins in B16 melanoma: of 963 non-synonymous somatic point mutations, 252 or 26% occurred in plasma membrane proteins (Castle, J. C. et al. 2012 Cancer Research 72(5): 1081-91).

If tumor mutations occur randomly in ECDs, will a sufficient number exist as linear epitopes amenable to use in peptide-based vaccines? Early studies, based on small numbers of Abs, suggested that the relative frequency of linear vs. conformational protein epitopes recognized by Abs was 1 in 10. Current estimates, based on vast HIV databases (HIV Molecular Immunology Database. Antibody Epitope Summary 2012. Available on the World-Wide-Web at: hiv.lanl.gov/content/immunology/tables/ab_summary.html), indicate that the prevalence of linear B cell epitopes in the ECD of glycoproteins may be significantly higher than that. In HIV gp160, the prevalence of linear B cell epitopes is 0.25, and in Tat it's 0.34, for human Ab responses induced by HIV infection and by immunization with HIV peptides (Sollner, J. and Mayer, B. 2006 J Molec Recog 19(3): 200-8).

Can linear B cell epitopes be predicted accurately? Algorithms to predict linear B cell epitopes have been in development for over three decades. Current open access machine learning algorithms have demonstrated reasonable accuracy (Sollner, J. and Mayer, B. 2006 J Molec Recog 19(3): 200-8; Su, C.-H. et al. 2012 PloS One 7(2):e30617; Wang, H. W. et al. 2011 Journal of Biomedicine & Biotechnology 2011:432830; Wang, Y. et al. 2011 BMC Bioinformatics 12: 251; Larsen, J. E. et al. 2006 Immunome Research 2:2; and Wee, L. J. et al. 2010 BMC Genomics 11 Suppl 4:S21). But, the overriding criterion for a B cell epitope is solvent accessibility.

Are there enough somatic mutations in tumors? Methylcholanthrene-induced tumors in mice (Matsushita, H. et al. 2012 Nature 482(7385):400-404) and tumor cell lines (Chang, H. et al. 2011 PloS One 6(6):e21097) have the highest number of msSNPs at ˜3000 per tumor. Next in frequency of msSNPs are carcinogen-induced human tumors such as non-small cell lung carcinoma (NSCLC) in smokers at 100-300 mutations per tumor (Pleasance, E. D. et al. 2010 Nature 463(7278):184-90; Lee, W. et al. 2010 Nature 465(7297):473-7; and Liu, P. et al. 2012 Carcinogenesis 33(7): 1270-6), UV-induced melanoma with ˜180 per tumor (Pleasance, E. D. et al. 2010 Nature 463(7278):191-6; and Wei, X. et al. 2011 Nature Genetics 43(5):442-6) and head and neck cancer in smokers with an average of 100 msSNPs per tumor (Stransky, N. et al. 2011 Science 333(6046):1157-60). This is followed by all other non-carcinogen-induced human tumors with a lower frequency of mutations.

In some embodiments, a gene containing an identified SNP is expressed at the 25th or higher percentile amongst all expressed genes in the patient. In other embodiments, the gene is expressed at or above the 5th, 10th, 20th, 30th, 40th, 50th, 60th, 70th, 80th, 90th or 95th percentile amongst all expressed genes in patient

We have set a minimum requirement of 5 effective targets for induction of autologous Ab-mediated tumor immunotherapy. Under immunologic pressure, a tumor can easily mutate away from 1 and possibly 2 target antigens, but evading 3 or more simultaneously is far less likely. If ˜26% of tumor mutations occur in plasma membrane proteins, of which ˜30% can function as linear B cell epitopes, then carcinogen-induced human lung tumors may provide 8 to 23 Ab-accessible target antigens per tumor, while melanoma may provide on average 14 effective targets for autologous Ab. The more immunogenic epitopes used, the better, because an individual's tumor can be genomically heterogeneous (Gerlinger, M. et al. 2012 The New England Journal of Medicine 366(10):883-92). Ab epitopes are more robust than T cell epitopes, because they cannot be evaded as an entire class the way cytotoxic T cell immunity can be escaped by loss of β2 microglobulin expression.

### EXAMPLE 1

Methods of Identifying Tumor-Specific, Somatically Mutated Sequences that are Antibody-Accessible and Immunogenic

The feasibility of Ab-based active tumor immunotherapy depends on the frequency of tumor-specific, somatically mutated sequences that are Ab-accessible and immunogenic. To assess this, we determined the frequency of somatic mutations in carcinogen-induced human lung tumors in smokers that would be expected to be immunogenic for antibody-mediated tumor immunity.

There are many publicly available data sets of high-throughput sequencing of human tumors. For this purpose, we utilized the NCI's The Cancer Genome Atlas (TCGA) database (The Cancer Genome Atlas. 2012. Available on the internet at: tcga-data.nci.nih.gov/tcga/tcgaHome2.jsp), which catalogues sequencing data of paired tumor and normal human cells for a wide range of major human tumors. The cancer data we had access to consisted of processed gene expression and single nucleotide polymorphism annotations for 563 lung adenocarcinoma (LUAD) patients. These data were held in a combination of text flat file formats and XML database format. Patients were de-identified through the use of barcodes for each data file. The barcodes came in two formats. The first, which was the original format used by the TCGA, was human readable, with all data files for a given patient having the same, matchable prefix. The newer format was randomized hash tags, for which we did not have the patient matching information; thus all data files identified via hash tags alone were discarded, as we were unable to confidently match the gene expression with the SNPs. The collation of the raw data led to a collection of 18 patients for whom we were able to collect and match gene expression and polymorphism profiles.

The genes which were quantified in the TCGA were those included in the NCBI Refseq database, mapped to the human genome build 37.2. Similarly, the polymorphisms were determined in reference to build 37.2. We applied a series of cutoffs to filter the gene/SNP pairs, to select for those of potential clinical significance. The filters were applied in the following order:
1. The gene containing the SNP is expressed at the 25th or higher percentile amongst all expressed genes in the patient.
2. The SNP is a missense mutation, rather than nonsense, synonymous or terminating, that is, it changes the amino acid sequence of the expressed protein, without truncating it.
3. The SNP occurs within the coding region of the host transcript.
4. The transcript codes for a likely transmembrane protein which contains a cellular membrane embedding sequence.
5. Upon embedding in the cell membrane, the mutated amino acid lies within an extracellular loop and is solvent-accessible.

The first filter was determined by counting all genes quantified per patient for which there was at least one RNA-Seq read. The second filter was based upon annotation provided in the TCGA SNP summary flat files, which identified missense polymorphisms. Note, we discarded indels, as they would result in frameshifts which would change the entire subsequent sequence. The SNPs were determined to be in coding regions by mapping the coordinates given in the flat files to those of CDS regions in the GTF file mapping of the refFlat table for Refseq. Thus, the first three filters were based solely upon experimentally verified annotations from reference databases.

The fourth and fifth filters were based upon bioinformatic prediction software. For transmembrane structure prediction, the CDS regions of the remaining transcripts were concatenated and translated into amino acid sequences, with the relevant amino acids mutated to reflect the missense SNPs. These sequences were fed into the TMHMM 2.0 web server. This algorithm predicts transmembrane helices, membrane embedding sequences, and transmembrane protein topology with a hidden Markov model, trained on known protein structures. We then selected those proteins that the algorithm predicted would have the mutation in an extracellular loop. Finally, for the remaining genes, we took just the relevant extracellular loop sequences, and applied a simply hydrophobicity analysis from the EMBOSS package, to confirm that the mutations were in solvent accessible regions.

TABLE 1
Examples of Single Nucleotide Polymorphism that Map to
Extracellular Domains of Expressed Membrane Proteins

| | | | |
|---|---|---|---|
| Host Gene | Host Chrom | Base (1-Based) | Mutation |

i. Patient ID: TCGA-44-5645

| | | | |
|---|---|---|---|
| CUX1 | chr7 | 101801866 | T |
| MAN2A1 | chr5 | 109091091 | G |
| SLC9A5 | chr16 | 67298944 | C |
| TLR6 | chr4 | 38830532 | A |

ii. Patient ID: TCGA-44-6146

| | | | |
|---|---|---|---|
| B3GNT1 | chr11 | 66114503 | T |
| CCL15 | chr17 | 34325389 | G |
| NTM | chr11 | 132016192 | T |
| RYR2 | chr1 | 237754106 | A |
| RYR2 | chr1 | 237829818 | T |
| UNC5CL | chr6 | 41002476 | A |

iii. Patient ID: TCGA-44-6147

| | | | |
|---|---|---|---|
| CD93 | chr20 | 23066732 | G |
| CDH6 | chr5 | 31299636 | A |
| CPT1A | chr11 | 68530172 | A |
| CSF2RB | chr22 | 37329941 | T |
| ENTPD7 | chr10 | 101458381 | A |
| LPPR4 | chr1 | 99771298 | T |
| LRIG1 | chr3 | 66436675 | C |
| NELL2 | chr12 | 45059372 | A |
| PARM1 | chr4 | 75937931 | A |
| ROBO2 | chr3 | 77629248 | A |
| RYR2 | chr1 | 237664020 | A |
| SNX13 | chr7 | 17838743 | G |
| TSNARE1 | chr8 | 143427140 | A |
| VSIG4 | chrX | 65252505 | A |

iv. Patient ID: TCGA-44-6148

| | | | |
|---|---|---|---|
| MUC4 | chr3 | 195505772 | G |

v. Patient ID: TCGA-44-6776

| | | | |
|---|---|---|---|
| EDA | chrX | 69255353 | T |
| EPHB2 | chr1 | 23111087 | A |
| FAT1 | chr4 | 187628565 | T |
| FRAS1 | chr4 | 79432632 | T |
| IL17RA | chr22 | 17589773 | T |
| KCNH2 | chr7 | 150644119 | C |
| KDR | chr4 | 55981449 | T |
| LDLRAD3 | chr11 | 36103274 | A |
| SLC6A8 | chrX | 152958581 | T |
| ST6GALNAC6 | chr9 | 130649020 | C |
| SYT1 | chr12 | 79679569 | A |
| TMTC4 | chr13 | 101308647 | A |
| TNC | chr9 | 117810558 | G |
| TNFRSF18 | chr1 | 1139804 | T |

vi. Patient ID: TCGA-44-6777

| | | | |
|---|---|---|---|
| ACACB | chr12 | 109613836 | T |
| AXL | chr19 | 41745172 | T |
| BAI2 | chr1 | 32221746 | C |
| BMP2 | chr20 | 6759046 | G |
| CD5 | chr11 | 60885907 | T |
| CD86 | chr3 | 121825068 | A |
| COLEC12 | chr18 | 321670 | A |
| EPHB1 | chr3 | 134880876 | G |
| EPHB6 | chr7 | 142563293 | A |
| FAIM3 | chr1 | 207087253 | T |
| FAM189B | chr1 | 155218083 | G |
| FAT4 | chr4 | 126242659 | T |
| FAT4 | chr4 | 126242685 | T |
| GPR124 | chr8 | 37688297 | T |
| ITGA1 | chr5 | 52145269 | T |
| ITGA2 | chr5 | 52369034 | A |
| ITGA4 | chr2 | 182339792 | A |
| ITGA8 | chr10 | 15646286 | A |
| ITGAL | chr16 | 30522247 | T |
| ITGAX | chr16 | 31373976 | T |
| ITPR2 | chr12 | 26774132 | A |
| LAG3 | chr12 | 6884556 | A |
| LMAN1 | chr18 | 57013159 | A |
| LPCAT1 | chr5 | 1494959 | A |
| MCAM | chr11 | 119183624 | A |
| MUC16 | chr19 | 9073651 | T |
| PCDHB7 | chr5 | 140554442 | T |
| PEAR1 | chr1 | 156877446 | A |
| PTGER4 | chr5 | 40692076 | A |
| PXDN | chr2 | 1652789 | T |
| RYR2 | chr1 | 237551419 | G |
| RYR2 | chr1 | 237806644 | T |
| SGMS2 | chr4 | 108817064 | T |
| SMCR7 | chr17 | 18167939 | G |
| STAB1 | chr3 | 52550187 | A |
| TLR1 | chr4 | 38799759 | G |

vii. Patient ID: TCGA-44-6778

| | | | |
|---|---|---|---|
| BTN3A2 | chr6 | 26373142 | T |
| C1orf27 | chr1 | 186367533 | G |
| CHST3 | chr10 | 73766983 | G |
| EDEM1 | chr3 | 5243547 | T |
| EPHB6 | chr7 | 142562205 | T |
| FAM20B | chr1 | 179013302 | G |
| FMO4 | chr1 | 171300844 | T |
| GLB1L2 | chr11 | 134239728 | T |
| GPRC5B | chr16 | 19884010 | T |
| GRAMD1B | chr11 | 123471176 | T |
| GRM6 | chr5 | 178417662 | T |
| LPCAT1 | chr5 | 1489886 | A |
| LRP4 | chr11 | 46905469 | A |
| 11-Mar | chr5 | 16067755 | G |
| NPFFR2 | chr4 | 73013164 | T |
| NRCAM | chr7 | 107824974 | T |
| P2RY8 | chrX | 1584526 | G |
| PCDHB2 | chr5 | 140474750 | T |
| PCDHGA4 | chr5 | 140736646 | G |
| PIGG | chr4 | 517555 | A |
| RYR2 | chr1 | 237729891 | T |
| SCNN1B | chr16 | 23360173 | C |
| SEMA5A | chr5 | 9197321 | T |
| SIGLEC5 | chr19 | 52130747 | A |
| SLC39A6 | chr18 | 33706670 | A |

viii. Patient ID: TCGA-49-4512

| | | | |
|---|---|---|---|
| CDH5 | chr16 | 66434763 | CDH5 |
| FRAS1 | chr4 | 79410160 | FRAS1 |
| TMEM5 | chr12 | 64202648 | TMEM5 |

ix. Patient ID: TCGA-49-6742

| | | | |
|---|---|---|---|
| CDH5 | chr16 | 66434763 | T |
| FRAS1 | chr4 | 79410160 | T |
| TMEM5 | chr12 | 64202648 | T |
| ATP11C | chrX | 138857035 | G |
| CDH19 | chr18 | 64218487 | C |
| CDH6 | chr5 | 31316422 | T |
| CELSR3 | chr3 | 48686568 | T |
| CELSR3 | chr3 | 48696534 | C |
| DSC2 | chr18 | 28660165 | T |
| FNDC3A | chr13 | 49752734 | C |
| LRRN1 | chr3 | 3888070 | C |
| MCTP2 | chr15 | 94858848 | T |
| NOTCH2 | chr1 | 120612013 | A |
| NRXN3 | chr14 | 79175708 | C |
| OSBPL8 | chr12 | 76763494 | A |
| PCDHA11 | chr5 | 140249821 | T |
| PLA2R1 | chr2 | 160884750 | A |
| PTCH1 | chr9 | 98242859 | A |
| RNASE10 | chr14 | 20978765 | T |
| RNF121 | chr11 | 71701662 | G |
| SCCPDH | chr1 | 246922379 | G |
| SLC25A39 | chr17 | 42398804 | A |
| SLC7A5 | chr16 | 87866630 | C |
| TM7SF3 | chr12 | 27128447 | T |
| VCAM1 | chr1 | 101186146 | A |
| WFS1 | chr4 | 6296864 | T |
| ZP3 | chr7 | 76058924 | C |

x. Patient ID: TCGA-49-6743

| | | | |
|---|---|---|---|
| ABCA10 | chr17 | 67197798 | T |
| ABCA2 | chr9 | 139909475 | C |
| ABCC3 | chr17 | 48741471 | T |
| ABCD4 | chr14 | 74757123 | T |
| BACE2 | chr21 | 42598273 | A |
| C1orf27 | chr1 | 186375307 | A |
| C20orf54 | chr20 | 745853 | A |
| C7orf58 | chr7 | 120906781 | T |
| CD33 | chr19 | 51728623 | A |
| CDH18 | chr5 | 19839061 | G |
| CYSLTR1 | chrX | 77529230 | T |
| DARC | chr1 | 159175928 | C |
| DENND1B | chr1 | 197480959 | A |
| ENPP1 | chr6 | 132168987 | T |
| FAM3C | chr7 | 121004205 | C |
| FAM3C | chr7 | 121011399 | T |
| FKRP | chr19 | 47259284 | T |
| FOLH1 | chr11 | 49168333 | A |
| FOLH1 | chr11 | 49179509 | A |
| GALNT10 | chr5 | 153765889 | T |
| ICAM3 | chr19 | 10446542 | A |
| IL12RB2 | chr1 | 67833639 | A |
| ITGA1 | chr5 | 52211397 | T |
| ITGA8 | chr10 | 15628602 | A |
| ITGAE | chr17 | 3663533 | A |
| JAG1 | chr20 | 10630973 | A |
| KCNMA1 | chr10 | 78771761 | T |
| KCNN3 | chr1 | 154687378 | A |
| KIAA1109 | chr4 | 123128714 | A |
| LILRB1 | chr19 | 55144158 | C |
| LRMP | chr12 | 25260973 | G |
| LRP1B | chr2 | 141819801 | C |
| LRP1B | chr2 | 141986830 | C |
| LRP2 | chr2 | 170048424 | T |
| LRRN2 | chr1 | 204587561 | G |
| LRRN2 | chr1 | 204587761 | T |
| MAN2A1 | chr5 | 109183385 | A |
| MGAM | chr7 | 141752635 | A |
| MGAM | chr7 | 141759384 | T |
| MUC16 | chr19 | 9076419 | A |
| NAALADL2 | chr3 | 174974218 | T |
| NAALADL2 | chr3 | 175184908 | G |
| NOX4 | chr11 | 89075295 | C |
| PCDHAC1 | chr5 | 140307901 | T |
| PCDHB7 | chr5 | 140552601 | A |
| PCSK5 | chr9 | 78804121 | C |
| PIGT | chr20 | 44049033 | C |
| PRTG | chr15 | 55965661 | C |
| PTCHD3 | chr10 | 27702206 | T |
| PVRIG | chr7 | 99818185 | A |
| RNF180 | chr5 | 63509778 | G |
| ROS1 | chr6 | 117681505 | T |
| RTN1 | chr14 | 60212963 | A |
| RYR1 | chr19 | 38990388 | A |
| RYR1 | chr19 | 39018312 | T |
| RYR2 | chr1 | 237753955 | C |
| RYR2 | chr1 | 237755119 | T |
| RYR2 | chr1 | 237802366 | T |
| RYR2 | chr1 | 237870252 | C |
| SDK1 | chr7 | 4150307 | A |
| SDK1 | chr7 | 4152959 | A |
| SIRPB2 | chr20 | 1459098 | A |
| SLC3A2 | chr11 | 62656092 | T |
| THSD7A | chr7 | 11501711 | A |
| TMEM30A | chr6 | 75969092 | A |
| TYRO3 | chr15 | 41860509 | T |
| TYRP1 | chr9 | 12704597 | G |

xi. Patient ID: TCGA-49-6744

| | | | |
|---|---|---|---|
| ABCG2 | chr4 | 89052998 | T |
| AFG3L2 | chr18 | 12353157 | A |
| ATP1B4 | chrX | 119505001 | G |
| CMKLR1 | chr12 | 108686090 | A |
| DCHS1 | chr11 | 6650685 | A |
| DNAJC14 | chr12 | 56221779 | A |
| EGFLAM | chr5 | 38425138 | A |
| EPHB6 | chr7 | 142564758 | T |
| FAT4 | chr4 | 126369691 | T |
| FRAS1 | chr4 | 79461787 | A |
| GPR125 | chr4 | 22425944 | C |
| HLA-DQA2 | chr6 | 32712989 | A |
| HMGCR | chr5 | 74655898 | T |
| ITGAM | chr16 | 31283276 | T |
| KIAA1324L | chr7 | 86526823 | G |
| LDLRAD3 | chr11 | 36057670 | C |
| LRRN3 | chr7 | 110763529 | C |
| MAN2A1 | chr5 | 109110522 | T |
| PCDHGA1 | chr5 | 140711999 | T |
| PIGM | chr1 | 160001510 | A |
| PTPRU | chr1 | 29606079 | T |
| RYR2 | chr1 | 237843805 | G |
| RYR2 | chr1 | 237886560 | A |
| SEMA4D | chr9 | 92002517 | T |
| SEMA5A | chr5 | 9063002 | T |
| STS | chrX | 7175602 | T |

xii. Patient ID: TCGA-49-6745

| | | | |
|---|---|---|---|
| CDHR3 | chr7 | 105662823 | C |
| CFTR | chr7 | 117234984 | C |
| CHRNB1 | chr17 | 7350939 | A |
| ITPR2 | chr12 | 26629850 | G |
| ITPR3 | chr6 | 33655052 | T |
| MUC16 | chr19 | 9047598 | G |
| PLA2R1 | chr2 | 160825826 | T |
| PTCH1 | chr9 | 98229610 | A |
| PTGER4 | chr5 | 40692192 | C |
| SMPD1 | chr11 | 6413070 | G |
| TSPAN31 | chr12 | 58140859 | T |

xiii. Patient ID: TCGA-50-5931

| | | | |
|---|---|---|---|
| ABCA13 | chr7 | 48312879 | T |
| ABCA13 | chr7 | 48337984 | C |
| BVES | chr6 | 105549026 | G |
| CHST10 | chr2 | 101014379 | A |
| CLCA4 | chr1 | 87040257 | A |
| COL14A1 | chr8 | 121262859 | G |
| FOLH1 | chr11 | 49204782 | A |
| GABBR1 | chr6 | 29595311 | A |
| GALNT6 | chr12 | 51773084 | A |
| GRM4 | chr6 | 34003675 | A |
| HEPH | chrX | 65415023 | A |
| HSD17B13 | chr4 | 88239557 | T |
| IFNGR2 | chr21 | 34793969 | T |
| IGSF3 | chr1 | 117122485 | A |
| LEPR | chr1 | 66038102 | T |
| MUC16 | chr19 | 9063480 | C |
| MUC16 | chr19 | 9091090 | T |
| NTM | chr11 | 132081940 | T |
| PCDHB3 | chr5 | 140480379 | T |
| RYR2 | chr1 | 237947531 | G |
| SLC9A6 | chrX | 135067822 | T |
| SPG7 | chr16 | 89613071 | T |
| TMEM14A | chr6 | 52548938 | A |
| VSIG4 | chrX | 65259825 | A |

xiv. Patient ID: TCGA-50-5932

| | | | |
|---|---|---|---|
| B4GALT6 | chr18 | 29206995 | A |
| EPHA3 | chr3 | 89448468 | A |
| GPR98 | chr5 | 90020785 | T |
| PCDHGA3 | chr5 | 140723683 | A |
| PTPRG | chr3 | 62189079 | T |

xv. Patient ID: TCGA-50-5935

| | | | |
|---|---|---|---|
| CYYR1 | chr21 | 27945234 | T |
| KIAA1109 | chr4 | 123268929 | A |
| P2RX5 | chr17 | 3593383 | C |
| PDE3B | chr11 | 14880641 | T |
| RYR1 | chr19 | 39018359 | A |
| SPG7 | chr16 | 89614444 | A |
| TNFRSF11A | chr18 | 60036591 | A |

xvi. Patient ID: TCGA-91-6829

| | | | |
|---|---|---|---|
| ABCA7 | chr19 | 1049420 | T |
| ABCB1 | chr7 | 87179246 | A |
| ABCB5 | chr7 | 20762679 | G |
| ABCC9 | chr12 | 21958120 | G |
| ABI3BP | chr3 | 100605099 | T |
| ASTN2 | chr9 | 120053691 | T |
| B4GALNT4 | chr11 | 376276 | T |
| BSG | chr19 | 581345 | G |
| CD248 | chr11 | 66084402 | A |
| CD99L2 | chrX | 149963906 | C |
| CDH23 | chr10 | 73455252 | T |
| CDH6 | chr5 | 31294121 | T |
| CLCN4 | chrX | 10181855 | G |
| CYP4V2 | chr4 | 187130045 | C |
| ELTD1 | chr1 | 79404923 | T |
| EMR2 | chr19 | 14877104 | T |
| FANCM | chr14 | 45668130 | C |
| FCGR1A | chr1 | 149761697 | G |
| FREM1 | chr9 | 14776091 | T |
| FREM2 | chr13 | 39438599 | C |
| GAL3ST4 | chr7 | 99757918 | T |
| GLT8D2 | chr12 | 104393245 | G |
| GPR125 | chr4 | 22422577 | T |
| GPR133 | chr12 | 131466608 | C |
| GPR133 | chr12 | 131490582 | T |
| HGF | chr7 | 81381537 | C |
| INSR | chr19 | 7184418 | A |
| KCNN4 | chr19 | 44273904 | T |
| LGI2 | chr4 | 25005746 | T |
| LGR6 | chr1 | 202245436 | T |
| LRP12 | chr8 | 105503299 | C |
| LRRC32 | chr11 | 76371892 | A |
| LYSMD4 | chr15 | 100269825 | T |
| MAN2A1 | chr5 | 109051926 | T |
| MARCO | chr2 | 119739773 | C |
| NDST2 | chr10 | 75563708 | A |
| NIPAL4 | chr5 | 156895745 | A |
| NRSN1 | chr6 | 24145935 | A |
| NTN4 | chr12 | 96131706 | G |
| PCDHB12 | chr5 | 140590281 | T |
| PCDHGA3 | chr5 | 140724427 | C |
| PRSS16 | chr6 | 27222627 | C |
| PTCRA | chr6 | 42893278 | A |
| PTPRO | chr12 | 15652394 | A |
| PXDN | chr2 | 1653374 | T |
| S1PR1 | chr1 | 101704852 | T |
| SC5DL | chr11 | 121174109 | C |
| SCG2 | chr2 | 224463114 | A |
| SLC12A9 | chr7 | 100451828 | A |
| SLC25A3 | chr12 | 98992384 | T |
| SLC4A2 | chr7 | 150771574 | G |
| SNX13 | chr7 | 17836494 | T |
| SNX14 | chr6 | 86238049 | G |
| SNX19 | chr11 | 130785484 | G |
| THSD7A | chr7 | 11418709 | A |
| TLR1 | chr4 | 38798855 | G |
| TMEM132A | chr11 | 60703723 | T |
| TMTC1 | chr12 | 29667422 | T |
| TRPC4 | chr13 | 38225534 | G |
| UXS1 | chr2 | 106717570 | C |
| VCAM1 | chr1 | 101198042 | T |

xvii. Patient ID: TCGA-91-6835

| | | | |
|---|---|---|---|
| CD1E | chr1 | 158325668 | A |
| CDH6 | chr5 | 31316327 | G |
| EGFR | chr7 | 55259515 | G |
| KLRK1 | chr12 | 10525829 | T |
| MUC16 | chr19 | 9088564 | T |
| SLC27A3 | chr1 | 153748206 | A |

xviii. Patient ID: TCGA-91-6836

| | | | |
|---|---|---|---|
| ABCB5 | chr7 | 20691133 | T |
| ABCC9 | chr12 | 21958111 | A |
| ABCG2 | chr4 | 89034589 | A |
| ABHD6 | chr3 | 58252959 | A |
| ACSL4 | chrX | 108906561 | A |
| CACNA1B | chr9 | 141006877 | T |
| CD163L1 | chr12 | 7522016 | T |
| CLDN4 | chr7 | 73245979 | T |
| ECM1 | chr1 | 150483594 | A |
| ESYT1 | chr12 | 56531422 | T |
| EVC | chr4 | 5785367 | A |
| FAT4 | chr4 | 126411157 | A |
| FCGR3A | chr1 | 161518373 | T |
| FLRT2 | chr14 | 86089362 | A |
| FLRT3 | chr20 | 14307564 | A |
| FNDC3B | chr3 | 171965470 | T |
| GCNT3 | chr15 | 59911449 | A |
| GIMAP1 | chr7 | 150417567 | T |
| GIMAP5 | chr7 | 150439681 | A |
| GRAMD1B | chr11 | 123465532 | A |
| HBEGF | chr5 | 139722344 | A |
| IGSF9 | chr1 | 159900160 | A |
| IL12RB2 | chr1 | 67796441 | T |
| ITGA1 | chr5 | 52145249 | T |
| ITGA9 | chr3 | 37670703 | A |
| ITGAL | chr16 | 30486638 | T |
| KIAA0319L | chr1 | 35972379 | A |
| KIAA1109 | chr4 | 123161126 | T |
| KIAA1161 | chr9 | 34372354 | A |
| L1CAM | chrX | 153133300 | T |
| LEPR | chr1 | 66081754 | G |
| LPHN2 | chr1 | 82409162 | A |
| LRP1 | chr12 | 57590052 | C |
| LRP10 | chr14 | 23344725 | A |
| LRRC4B | chr19 | 51021718 | T |
| NPTXR | chr22 | 39224385 | A |
| NTN4 | chr12 | 96107010 | A |
| PCDHB11 | chr5 | 140579964 | A |
| PCDHB12 | chr5 | 140588603 | T |
| PCDHB4 | chr5 | 140501701 | C |
| PCDHB9 | chr5 | 140568789 | T |
| PCDHGB4 | chr5 | 140768013 | T |
| PLXDC1 | chr17 | 37296055 | T |
| PLXNA2 | chr1 | 208218542 | T |
| PTPRN | chr2 | 220172515 | A |
| PXDN | chr2 | 1670144 | A |
| SLC12A6 | chr15 | 34531187 | T |
| SLC12A6 | chr15 | 34531252 | A |
| SLC39A1 | chr1 | 153935099 | A |
| SLC39A6 | chr18 | 33706813 | A |
| SLC6A17 | chr1 | 110737248 | T |
| SLMAP | chr3 | 57898147 | T |
| TBL2 | chr7 | 72987710 | T |
| THSD7A | chr7 | 11464362 | A |
| TM2D3 | chr15 | 102182797 | A |
| TM9SF1 | chr14 | 24663940 | T |
| TMED1 | chr19 | 10945774 | A |
| TMEM182 | chr2 | 103380886 | T |
| TMEM2 | chr9 | 74319588 | A |
| TMEM74 | chr8 | 109796659 | A |
| TMTC3 | chr12 | 88570021 | T |
| TNFSF14 | chr19 | 6665094 | A |
| TRPM4 | chr19 | 49661459 | A |

The number of Refseq genes analyzed was 39932. The mean number SNPs was 442, the mean number of missence Single Nucleotide Polymorphisms (MS-SNPs) was 286; the Mean number of 25th percentile SNPs was 170; the mean number of 25th percentile CDS SNPs was 160 and the mean number of target SNPs per patient was 27. “1-Based” in column 3 of the above data refers to the SNP locus being specified along the chromosome with the first base of the chromosome being numbered 1; in some instances, the first base of a gene in the database is numbered 0. Thus, specifying “1-Based” resolves that ambiguity. Fifteen out of eighteen patients, or 83%, had 6 or more target SNPs.

These data confirm that the approach disclosed herein of using somatic mutations in the extracellular domain (ECD) of plasma membrane proteins on tumor cells as the source of immunogens for active induction of tumor-specific Ab-mediated immunotherapy is highly feasible.

Given the feasibility of this approach, preclinical testing is straightforward. We describe in detail two approaches in Examples 2 and 3.

### EXAMPLE 2

Identification of the Somatically Mutated Sequences in C57BL Lewis Lung Carcinoma that are Potential Antibody Targets

The C57BL Lewis lung carcinoma (CRL-1642), B16-F10 melanoma (CRL-6475) and EL4 thymoma (TIB-39) is obtained from ATCC. The cell lines are expanded in culture according to ATCC protocols and sufficient aliquots are frozen to ensure that all experiments are done with the same pool of cells, to keep genomic heterogeneity to a minimum. Whole exome capture sequencing (WECS) is done on Lewis lung carcinoma cells and B6(Cg)-Tyrc-2J/J (B6-albino) lung using standard protocols (Castle, J. C. et al. 2012 Cancer Research 72(5):1081-91). B6-albino mice have a spontaneous mutation in the tyrosinase gene and are preferable to pigmeted mice for in vivo bioluminescence imaging of tumor growth. One would not expect Lewis lung carcinoma cells to express tyrosinase, but if they do, all experiments can be done with C57BL/6 mice instead. Genomic DNA for WECS are prepared from LN2 frozen samples using Qiagen DNeasy Blood and Tissue Kit. Total RNA for RNA-Seq are extracted using Trizol and Qiagen RNeasy Mini Kit. B6-albino LN2 frozen tissue are first pulverized using an LN2 cooled motorized CryoGrinder prior to extraction of DNA. DNA and RNA samples are pre-analyzed by NanoDrop and their quality determined by Agilent Bioanalyzer at San Diego Veterans Medical Research Foundation GeneChip Core. Acceptable samples are sent to EdgeBio or OtoGenetics for Exome Capture Sequencing, using Agilent Sure SelectXT Mouse All Exon Kit and Illumina HiSeq 2000, paired end sequencing at >50x coverage, with FASTQ files obtained. Three Lewis lung samples and 2 B6-albino samples are run for WECS and three Lewis lung samples for RNA-Seq.

Preprocessing of the FASTQ files (Cock, P. J. et al. 2010 Nucleic Acids Research 38(6):1767-71) is done using the FASTx-Toolkit (cancan.cshl.edu. FASTX-Toolkit 2009. Available on the internet at: cancan.cshl.edu/labmembers/gordon/fastx_toolkit/), which enables adaptor and barcode removal, quality format conversion, and filtering of reads based upon quality scores. Alignment to the genome is done with Bowtie (Langmead, B. et al. 2009 Genome Biology 10(3):R25), designed specifically for high-throughput sequencing data. The package Samtools (Li, H et al. Bioinformatics 25: 2078-2079) is used for Bowtie output processing and SNP identification. Statistical modeling and selection of SNPs in comparison to a reference sample, as well as filtering of SNPs that are likely to be the result of sequencing errors is done using SAMtools. SNP selection can also be done with MutationSeq (Ding, J. et al. 2012 Bioinformatics 28(2):167-75) and the ATLAS2 Suite (Challis, D. et al. 2012 BMC Bioinformatics 13:8). DNA mutations are considered validated if confirmed by RNA-Seq reads. For RNA-Seq data, localization of SNPs to exons is done using BEDTools (Quinlan, A. R. and hall, I. M. 2010 Bioinformatics 26(6):841-2) and Cufflinks software (Trapnell, C. et al. 2010 Nature Biotechnology 28(5):511-5) is used for expression quantification.

SNP localization is ascertained based on terminal sequences that identify membrane insertion and targeting to the plasma membrane rather than internal ones, e.g., TargetP (74). Orientation is determined by the location of the insertion sequence, and the pattern of transmembrane alpha helices, while solvent accessibility is predicted by the electronic properties of the amino acid sequence, e.g., NetSurfP (CBS Prediction Servers 2012. Available on the World-Wide-Web at: cbs.dtu.dk/services/). A wide range of software tools is available for each of these analyses. The identified target proteins are annotated from the UNIPROT (The UniProt Consortium 2012 Nucleic Acids Research 40(D1):D71-D5) and PFAM (Punta, M. et al. 2012 Nucleic Acids Research 40(D1):D290-D301) databases, to identify documented extracellular structures. If a candidate protein has no known subcellular localization, WOLF-PSort (WoLF PSORT Protein localization predictor 2010. Available on the internet at: wolfpsort.org/aboutWoLF_PSORT.html.en) is used to predict plasma membrane localization based upon membrane-embedding signal motifs, amino acid content, and comparison against annotated structures. For those proteins identified by either annotation or computation to be likely embedded in the plasma membrane, the package TMHMM (TMHMM Server v. 2.0 Prediction of transmembrane helices in proteins 2012. Available on the World-Wide-Web at: http://www.cbs.dtu.dk/services/TMHMM/) is used to predict alpha helices in protein sequences. Based upon these predictions and the presence of the insertion sequence, ECDs are identified with high fidelity. These tumor-specific SNPs are further analyzed using three support machine vector (SMV)-based linear B cell epitope prediction algorithms (Wang, H. W. et al. 2011 Journal of Biomedicine & Biotechnology 2011:432830; Wang, Y. et al. 2011 BMC Bioinformatics 12: 251; and Wee, L. J. et al. 2010 BMC Genomics 11 Suppl 4:S21).

For further analysis, the predicted mutations are limited to one per protein and prioritized by selection consensus, rank (if available), and high expression in the tumor. At this point, false positives are more acceptable than false negatives, because false positives can be screened out.

### EXAMPLE 3

Determination of Whether the Somatic Mutations Selected Above are: a) Immunogenic in B6 Mice, b) Induce Antibodies that Recognize the Intact Protein on the Surface of the Tumor Cells, and/or c) Retard the Growth Of the Tumor In Vivo.

The Lewis lung carcinoma somatic mutations selected above is further evaluated for potential immunogenicity based on the extent to which the chemical character of the substituted amino acid differs from that of the wild-type amino acid. the optimal length of each mutated peptide sequence, the starting and ending amino acids, and the optimal method for covalent attachment of the peptide to tetanus toxoid, the most commonly used carrier protein in U.S. licensed conjugate vaccines.

The mutated peptides, ±20 residues long, are synthesized with >95% purity by Think Peptides or PolyPeptide, and conjugated to tetanus toxoid by MDS, EDC or activated EDC, depending on the sequence of the peptide and the position of the peptide in the protein sequence. Each conjugate preferably contains only one peptide. Peptide solubility is enhanced by selecting hydrophilic sequences likely to be exposed on the solvent face. Self-tolerance should prevent most auto-Ab formation, but the potential for this can be monitored closely in immunized mice, while Abs that fail to recognize the mutated tumor peptide will be irrelevant. Conjugation ratios are preferably 1:2 to 2:1 peptide to toxoid w:w ratios, corresponding to comparable ratios found in licensed tetanus toxoid conjugate vaccines. Unbound peptides are removed by dialysis.

Groups of B6-albino mice are immunized i.d. on day 0 and 21 with 25 microg. of a single peptide:tetanus toxoid conjugate, incorporating an inflammasome activator (Alhydrogel) and a TLR9 agonist (CpG DNA), and accompanied by a proprietary topical adjuvant that promotes Abs of all IgG isotypes, as well as secretory IgA Abs. These conditions induce high levels of long-lived circulating and secretory Abs that can bind metastatic tumor cells in a variety of locations. The most high-titered, long-lived Ab is induced by exposure to live microorganisms (Amanna, I. J. et al. 2007 The New England Journal of Medicine 357(19):1903-15) that express multiple TLR and NLR agonists. Serum and BAL from individual animals can be obtained on day 42. Assays are run to detect Abs that specifically bind to the surface of Lewis lung carcinoma cells, accomplished by indirect immunofluorescent flow cytometry (FACS) using PE-anti-mouse IgG and PE-anti-mouse IgA, with specificity determined using B16 melanoma and EL4 thymoma cells.

Variable antibody responses and antigen binding are observed. This is consistent with the finding that numerous msSNPs in the B 16 melanoma are immunogenic for T cells in B6 mice (Castle, J.C. et al. 2012 Cancer Research 72(5):1081-91). Second, there are countless examples of Abs that distinguish single amino acid differences in an epitope; most recently and directly relevant, a SNP in the ECD of EGFR in a colorectal tumor resulted in resistance to cetuximab but not panitumumab, even though both mAbs block growth factor binding (Montagut, C et al. 2012 Nature Medicine 18(2):221-3). Sera from peptide conjugates that consistently fail to induce tumor-cell reactive Abs are subjected to peptide-specific ELISA. This identifies whether a peptide was not immunogenic, or it was, but the Ab did not detect the mutation on the surface of the tumor. This information identifies predictive algorithms that are the weakest and need to be made more stringent or eliminated. The influence of the chemical difference between the wild-type and the substituted amino acid in the mutated peptide is also discerned. This information enables selection of the best algorithms and ranking of the relative importance of each predictive factor.

The most direct test of the therapeutic potential of solvent accessible msSNPs, as a source of immunogens, is active immunization with the mutated peptides, confirmed to be immunogenic in the preceding experiments, followed by inoculation with tumor cells. Therapeutic tumor immunity experiments is not feasible in mice because high affinity IgG Abs can take 28-42 days to mature. Mice are so small that a growing tumor can destroy essential organ function before induced Abs can destroy the tumor. In humans, the window of opportunity is orders of magnitude greater. Mice are immunized twice i.d., as described above, but with a mixture of two conjugated peptides per site and a total of eight sites; one on either side of the lower abdomen and one on either flank to avoid antigenic competition, which can occur at the level of DCs in an individual draining lymph node. A small serum sample is obtained on day 42 for FACS analysis.

Luciferase transduced Lewis lung carcinoma cells (LL/2-luc-M38) obtained from Caliper Life Sciences are tested for the same level of surface expression of each mutated antigen as that on the untransduced cells. This is done by FACS analysis using the B6 peptide-specific antisera. For assessment of tumor growth, LL/2-luc-M38 cells are injected into the lateral tail vein of immunized and unimmunized control mice. For intravital bioluminescence imaging at 4 hours and 1, 2, and 7 days after tumor-cell injection, luciferin is injected i.p, and light emission is measured 5 minutes later. A final imaging is done between 14 and 21 days, prior to euthanasia for histologic examination. Bioluminescence imaging of live animals is performed on a Xenogen IVIS Lumina II optical imaging system (Caliper Life Sciences). The system has an adjustable 5×5 cm to 12×12 cm field of view, 50 to 400 μm (depending on zoom) spatial resolution, and a sensitivity of 100 photons/second/cm2, which corresponds to the detection of as few as 2,000 cells near the surface of an animal. A typical 1- to 5-min scan captures bioluminescence images from up to 3 mice placed side by side. Image analysis is performed with LIVING IMAGE™ software on a PC computer and the light signal quantified to compare tumor burden at different time points and between experimental groups based on photon flux (photons/second/cm2). The specificity control is luciferase transduced EL4 cells inoculated i.v.

If peptide immunization retards or prevents tumor growth, the minimum number of immunogenic peptides required for tumor protection is determined. Some of the peptide:TT conjugates may elicit T cell immunity specific for the mutated peptide. Therefore, confirmation that the observed protective immunity is Ab-mediated is obtained by passive transfer of the tumor-binding peptide-specific antisera obtained above, followed by challenge with LL/2-luc-M38 tumor cells and in vivo bioluminescence imaging.

The preceding Examples provide an experimentally determined frequency of tumor msSNPs, predicted to be Ab accessible, that are a) immunogenic in syngeneic mice, b) induce Abs that bind the mutated protein on the surface of intact tumor cells, and c) retard the growth of that tumor in syngeneic mice in vivo These methods provide a basis for the identification of immunogenic somatic mutations in a patient's tumor that can be used to construct an Ab-based therapeutic cancer vaccine that is non-toxic, cost effective, targets multiple different tumor antigens, and is able to penetrate the immunosuppressive tumor microenvironment, without the need to override normal immune controls.

While the present invention has been described in some detail for purposes of clarity and understanding, one skilled in the art will appreciate that various changes in form and detail can be made without departing from the true scope of the invention. All figures, tables, and appendices, as well as patents, applications, and publications, referred to above, are hereby incorporated by reference.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described herein.

In addition, the materials, methods, and examples described herein are illustrative only and are not intended to be limiting.

What is claimed is:

1. A method of identifying a tumor-specific antigen, the method comprising:
(a) identifying a tumor-specific mutation in an expressed gene of a subject having cancer,
(b) determining that said mutation is in a region of an open reading frame predicted to encode an extracellular domain of a membrane protein; and
(c) identifying a corresponding mutant polypeptide encoded by the expressed gene comprising the tumor-specific mutation.

2. The method of claim 1, wherein the mutation is a single nucleotide polymorphism (SNP).

3. The method of claim 1, wherein the mutant polypeptide is about 6-10 amino acids in length.

4. The method of claim 1, wherein the mutant polypeptide is greater than 10 amino acids in length.

5. The method of claim 1, wherein the mutant polypeptide is greater than 15 amino acids in length.

6. The method of claim 1, wherein the mutant polypeptide is greater than 20 amino acids in length.

7. The method of claim 1, wherein the mutant polypeptide is greater than 30 amino acids in length.

8. The method of claim 1, further comprising selecting a polypeptide identified in step (c) that serves as a B cell epitopes for the induction of autologous tumor-specific antibodies.

9. A method of inducing a tumor specific immune response in a subject comprising administering one or more polypeptides identified according to claim 1 to said subject.

10. The method of claim 9, further comprising administering an adjuvant to said subject.

11. A pharmaceutical composition comprising the mutant polypeptide identified according claim 1 and a pharmaceutically acceptable carrier.
